(19) United States
(12) Patent Application Publication
(10) Pub. No.: US 2010/0268181 A1
(43) Pub. Date: Oct. 21, 2010

(54) Superabsorbent Foam with Graphics on the Surface

(75) Inventors: Antje Ziemer, Mannheim (DE); Ernst Jürgen Bauer, Ludwigshafen (DE)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: BASF SE

(21) Appl. No.: 12/741,354

(22) PCT Filed: Nov. 10, 2008
(86) PCT No.: PCT/EP08/65195

§ 371 Date: May 4, 2010

(30) Foreign Application Priority Data
Nov. 15, 2007 (EP) 07120800.3

(51) Int. Cl. A61F 13/53; B32B 3/12; B29C 44/02

(52) U.S. Cl. 604/369; 428/159; 264/41

(57) ABSTRACT

A superabsorbent foam which comprises at least 1% by weight, based on the total weight of the dry foam, of inorganic pulverulent solid and bears graphics on at least one of its surfaces is obtainable by a process in which a foamed monomer mixture which comprises an appropriate amount of inorganic solid is polymerized in a mold which bears graphics on at least one inner surface.

The invention relates to a superabsorbent foam which has graphics on the surface, and to a process for producing it and to its use.

Superabsorbents are known. For such materials, names such as “high-swellability polymer”, “hydrogel” (often also used for the dry form), “hydrogel-forming polymer”, “water-absorbing polymer”, “absorbent gel-forming material”, “swellable resin”, “water-absorbing resin” or the like are also used. The substances in question are crosslinked hydrophilic polymers, especially polymers of (co)polymerized hydrophilic monomers, graft (co)polymers of one or more hydrophilic monomers on a suitable graft base, crosslinked cellulose ethers or starch ethers, crosslinked carboxymethyl cellulose, partly crosslinked polyalkylene oxide or natural products which are swellable in aqueous liquids, for example, guar derivatives, of which water-absorbing polymers based on partly neutralized acrylic acid are the most widespread. The essential properties of superabsorbents are their abilities to absorb several times their own weight in aqueous liquids and not to release the liquid again even under a certain pressure. The superabsorbent, which is usually used in the form of a dry powder, but is also known in the form of foams, is converted to a gel when it absorbs liquid, and correspondingly to a hydrogel (which in this case is indeed a hydrogel) when it absorbs water. Crosslinking is essential for synthetic superabsorbents and is an important difference from customary pure thickeners, since it leads to the insolubility of the polymers in water. Soluble substances would not be usable as superabsorbents. By far the most important field of use of superabsorbents is the absorption of body fluids. Superabsorbents are used, for example, in diapers for infants, incontinence products for adults or feminine hygiene products. Other fields of use are, for example, as water-retaining agents in market gardening, as water stores for protection against fire, for liquid absorption in food packaging, or quite generally for absorbing moisture.

Superabsorbent foams, i.e. water-absorbing foams based on crosslinked monomers comprising acid groups or based on crosslinked basic polymers are likewise known. Superabsorbent foams can be used, for example, as a liquid storage layer in hygiene articles or generally for the absorption, conduction or storage of aqueous liquids.

Processes for producing superabsorbents are also known. Superabsorbents based on acrylic acid, which are the most common on the market, are produced by free-radical polymerization of acrylic acid in the presence of a crosslinker (the “internal crosslinker”), and the acrylic acid is neutralized to a certain degree before, after or partly before and partly after the polymerization, typically by adding alkali, usually an aqueous sodium hydroxide solution. The internal crosslinker is typically a compound having at least two polymerizable groups which are polymerized into different polymer chains formed from the acrylic acid monomers in the polymerization and thus crosslink the polymer chains to one another. The polymer gel thus obtained is comminuted (according to the polymerization reactor used, this can be done simultaneously with the polymerization) and dried. The dry powder thus obtained (the “base polymer”) is typically crosslinked on the surface of the particles by reacting it with further crosslinkers which can form bonds between different functional groups of the polymer, for instance organic crosslinkers or polyvalent cations, for example, aluminum, (usually used in the form of aluminum sulfate), in order to obtain a more highly crosslinked surface layer compared to the particle interior.

Processes for producing superabsorbent foams are likewise known. Typically, a mixture comprising the monomer is foamed. This can be done by mechanical dispersion of gas bubbles, for example by beating gas in or injecting gas and decompression through a die, but also by decomposition of a gas-forming blowing agent in the monomer solution. The mixture thus foamed is then polymerized and optionally aftertreated.

Fredric L. Buchholz and Andrew T. Graham (eds.) give, in: “Modern Superabsorbent Polymer Technology”, J. Wiley & Sons, New York, U.S.A./Wiley-VCH, Weinheim, Germany, 1997, ISBN 0-471-19411-5, a comprehensive overview of known processes for producing superabsorbents and of superabsorbent foams.

Examples of superabsorbent foams and processes for their production are, for instance, WO 97/17 397 A1, WO 97/31971 A1, WO 99/44648 A1 and WO 00/52087 A1. These foams are produced by foaming a polymerizable aqueous mixture which comprises monoethylenically unsaturated monomers which comprise acid groups and have been neutralized to an extent of at least 50 mol %, crosslinkers and at least one surfactant, and subsequent polymerization of the foamed mixture. The polymerizable mixture is foamed by dispersing fine bubbles of a gas which is inert toward free radicals or by dissolving such a gas under elevated pressure in the polymerizable mixture and decompressing the mixture. The water content of the foams is adjusted to from 1 to 60% by weight. The foams can optionally be subjected to a surface postcrosslinking by spraying a crosslinker onto the foamed material or immersing the foam therein and heating the foam laden with crosslinker to a higher temperature. The foams are used, for example, in hygiene articles for acquisition, distribution and storage of body fluids. Carboxymethylcellulose, hydroxyethylcellulose, hydroxymethylcellulose, hydroxypropylcellulose and cellulose mixed ethers are disclosed as thickeners in these applications. It is also known to consolidate the foam by adding fine superabsorbent particles. Foams are often stabilized against fracture or cracking by adding fibers.

WO 03/06 6716 A1 discloses foams formed from water-absorbing basic polymers which are obtainable by foaming an aqueous mixture which comprises at least one basic polymer, such as polyvinylamine and at least one crosslinker such as glycidyl ether, and subsequently crosslinking the foamed mixture.

WO 03/066717 A2 discloses a process with which, by virtue of the addition of polymers containing amino groups, the wet strength of superabsorbent foams is increased and the residual monomer content is lowered.

WO 2004/007598 A1 discloses water-absorbing foams which, on the surface, have finely divided hydrophilic silicon dioxide and/or a surfactant. WO 2006/106108 A1 discloses foams which, as a result of treatment with a swelling retardant, absorb more slowly than conventional foams.

WO 2004/035668 A2 discloses water-absorbing foams which comprise superabsorbent fibers or fruit fibers, especially apple fibers. WO 2006/094977 A2 describes water-absorbing foams which comprise wood fibers or waste paper fibers.

It is also known that inorganic solids can be added to particulate superabsorbents as fillers. In particular, clay minerals are used for this purpose.

GB 2 082 614 A discloses a blend of superabsorbent powder and a filler selected from uncrosslinked cellulose derivatives, starch, particular clays and minerals, or mixtures thereof. The mixture has a higher absorption capacity than the calculated sum of the constituents. U.S. Pat. No. 4,500,670 teaches a mixture of superabsorbent and inorganic, water-insoluble powders, in which the inorganic powder improves the stiffness of the swollen gel. In the suspension polymerization process taught by U.S. Pat. No. 4,735,987 a highly expanding and gas-permeable superabsorbent is obtained by crosslinking superabsorbent particles with one another in suspension, the presence of an inorganic filler, for instance hydrotalcite, montmorillonite, talc, pyrophyllite or kaolinite being required. U.S. Pat. No. 4,914,066 discloses shaped bodies composed of bentonite comprising from 0.5 to 15% by weight of superabsorbent.

According to the teaching of WO 91/12 029 A1, WO 91/12 031 A1 or EP 799 861 A1 water-insoluble zeolites or activated carbon are used as an additive to superabsorbents in order to confine development of unpleasant odors. According to WO 01/13 965 A1 silicon-rich zeolites are used for this purpose.

U.S. Pat. No. 5,419,956 discloses absorbent articles which comprise superabsorbents to which, to improve the liquid distribution, inorganic powders such as silicon dioxide, aluminum oxide, titanium dioxide or clays, for example, kaolin or montmorillonite, are added. WO 01/68 156 A1 describes superabsorbents to which, both to improve the liquid conductivity and to bind unpleasant odors, alumina silicates, especially those with sheet structures such as saponite or montmorillonite, are added.

U.S. Pat. No. 3,900,378 teaches, in the production of a superabsorbent by crosslinking soluble polymers by means of ionizing radiation, the addition of a filler as a dispersant to the polymer particles. Examples of fillers also include minerals such as perlite, kieselguhr, clays, fly ash and magnesium silicates. According to the teaching of U.S. Pat. No. 5,733,576 such fillers can be added in the production of a superabsorbent which is a mixture of crosslinked polyacrylate and polysaccharide.

U.S. Pat. No. 6,124,391 discloses the use of inorganic powders, especially clays such as kaolin, as a means of counteracting the caking tendency of superabsorbents.

WO 00/72 958 A1 describes the use of clays as synergistic fillers of superabsorbents. WO 01/32 117 A1 teaches the use of hydrotalcite as a basic filler in a slightly acidic superabsorbent, in order to increase its tolerance towards sodium chloride.

It is an object of the invention to find a superabsorbent foam which has graphics on its surface, and a process for the production thereof. Accordingly, a superabsorbent foam has been found, which comprises at least 1% by weight, based on the total weight of the dry foam, of inorganic pulverulent solid and bears graphics on at least one of its surfaces. The inventive foam is obtainable by a process in which a foamed monomer mixture which comprises at least 1% by weight, based on the total weight of the finished dry superabsorbent foam, of inorganic pulverulent solid is polymerized in a mold which bears graphics on at least one internal surface.

In the context of this invention, “surface” of the foam is understood to mean the geometric surface of a foam molding, not the entire inner surface area of all pores of the foam, as might be determined, for example, by the known processes for measuring adsorption isotherms.

Graphics are understood to mean all kinds of symbols and patterns which can be represented on an essentially flat or flat surface by points, lines, areas, filling patterns or other graphic design elements, especially characters, letters, drawings, geometric, representational or abstract patterns and figures. A true three-dimensional design of a foam molding which can be obtained in a simple manner by corresponding three-dimensional geometric design of the mold in which the foam is polymerized is not what is meant. What is meant by an essentially flat surface is that the surface imparts an essentially flat appearance, but the graphics can retreat somewhat behind the surface of the foam or protrude therefrom. The symbols may thus quite possibly be perceptible to the touch, even though the three-dimensionality, just like in printing processes on paper, is typically minimal.

Superabsorbent foams are known from the prior art. According to the present invention, superabsorbent foam is understood to mean a foam which has a centrifuge retention capacity (“CRC”, test method described below in “determination methods” section) of at least 3 g/g, preferably at least 4 g/g, more preferably at least 5 g/g, especially at least The inventive superabsorbent foams comprise at least 1% by weight, based on the total weight of the dry foam, of inorganic pulverulent solid. In connection with superabsorbent foams, this is often also referred to as “filler”. Superabsorbent foams comprising fillers are known.

Like particulate superabsorbents, superabsorbent foams also usually have a finite water content. Drying to a residual water content which is no longer measurable is costly and inconvenient, and superabsorbents draw moisture from the environment. Often, the water content in the foam is deliberately adjusted to a particular desired value, generally to at least 1% by weight, preferably at least 2% by weight and more preferably to at least 3% by weight, based in each case on the total weight of the moistened foam. An upper limit for the water content ultimately arises merely through economic considerations with regards to the typical use of the foams for absorbing liquids, since the absorption capacity falls correspondingly with rising water content. Usually, a water content in the foam—before its use for liquid absorption—of not more than 50% by weight, preferably not more than 30% by weight and more preferably of not more than 20% by weight is established, all based on the total weight of the moistened foam. Typical water contents of foams are, for example, 5% by weight or 10% by weight, based in each case on the total weight of the moistened foam. However, superabsorbents feel dry even in the case of high water content. The water absorption is reversible. In order to create a defined basis for calculation of percentages, quantitative data in the context of this application, unless stated otherwise, are based on a dry superabsorbent foam, i.e. a superabsorbent foam which comprises no water. For a real foam which has a particular water content, conversion should be done correspondingly. The water content (also referred to as “moisture content” or “residual moisture”) is determined by the method specified below under “determination methods”.

In the context of this invention, data in ° A by weight are always—and also for constituents other than water—based on the total weight of the dry superabsorbent foam, unless explicitly stated otherwise in the individual case.

The inorganic pulverulent solid is a filler whose property of forming graphics on the surface in the course of polymerization is essential for the success of the process according to the invention and the suitability of a monomer mixture. In principle, any inorganic powder is suitable.

The size of the individual particles in the powder is typically generally at least 1 μm, preferably at least 5 μm and more preferably at least 10 μm, and generally at most 1000 μm, preferably at most 900 μm and more preferably at most 850 μm. The powder particles may also be aggregates or agglomerates of smaller primary particles. It is equally possible to use agglomerates which decompose to smaller particles only in the course of production of the monomer mixture, for instance in order to avoid problems with dust formation or formation of respirable dusts.

The inorganic powder is a particulate solid. Examples of such solids are oxides, clays, zeolites, inorganic pigments, minerals or generally solid, chemically inert substances (i.e. substances which do not significantly impair the polymerization or the use of the foam).

Suitable oxides are the metal oxides of groups 2 to 14 of the Periodic Table of the Elements, including the lanthanides and actinides. Examples of particularly suitable metal oxides are magnesium oxide, calcium oxide, strontium oxide and barium oxide, titanium dioxide, zirconium dioxide, vanadium oxide, chromium oxide, molybdenum oxide and tungsten oxide, manganese oxide, iron oxide, cobalt oxide, nickel oxide, copper oxide, zinc oxide, boron oxide, aluminum oxide, silicon dioxide, tin oxide, lead oxide, lanthanum oxide or cerium oxide. For clarification: the use of these common names is not intended to make any definitive statement regarding the valency of the metal in the oxide, i.e. regarding the stoichiometric composition of the oxide. When more than one oxide of an element is known, the use of all known oxides is generally possible. The selection is made according to considerations specific to the individual case, for instance according to the costs of an oxide, or its toxicity, stability or else color. Examples of very suitable oxides are titanium dioxide, especially in the rutile or anatase polymorphs, or silicon dioxide, prepared by precipitation or by pyrolytic means.

Clays are water-swellable silicate or aluminosilicate minerals which are typically degraded as sedimentary rock and are in some cases after-treated. However, they can also be produced synthetically. Examples are especially kaolinite, especially in the form of kaolin, illite, attapulgite (alternative name: palygorskite), sepiolite, montmorillonite, especially in the form of bentonite, pyrophyllite, saponite or talc. Other sheet silicates or sheet aluminosilicates can also be used, for example vermiculite or hydrotalcite.

Examples of other usable inorganic solids are sulfates such as magnesium sulfate or barium sulfate, carbonates such as calcium carbonate or magnesium carbonate or dolomite, silicates such as calcium silicate or magnesium silicate, carbides such as perlite or silicon carbide, diatomaceous earth or fly ash.

It is also possible to use mixtures of two or more of these solids.

In the production of superabsorbent foams from a monomer mixture comprising acrylic acid, some solids, especially carbonates decomposable by acid, such as calcium carbonate, can simultaneously also be used for foam formation. In this case, it should be ensured that the reaction parameters and the composition of the substance mixture are selected such that, in spite of the decomposition of the carbonate with foam formation, a sufficient amount of particulate solid remains.

The content of inorganic powder in the superabsorbent foam is generally at least 1% by weight, preferably at least 2% by weight and more preferably at least 5% by weight and generally at most 50% by weight, preferably at most 40% by weight and more preferably at most 20% by weight. This upper limit is, however, determined less by any influence on the ability of the foam to form graphics than by the desired absorption capacity of the foam for liquids, which is naturally lowered by non-superabsorbent fractions in the foam. If comparatively low absorption capacity of the foam can be tolerated or is even desired, the proportion of the inorganic powder may also be above the upper limits specified. The optimal powder content of the foam for the ability to form graphics depends on the specific powder and can be determined easily with a few routine tests. For example, the ability of a foam comprising kaolin to form graphics is optimal from a kaolin content of at least 10% by weight, even better of at least 20% by weight, in the case of a talc-containing foam at a talc content of at least 20% by weight, even better at least 40% by weight and in the case of a titanium dioxide-containing foam at a titanium dioxide content of at least 1% by weight, even better at least 5% by weight.

The inventive superabsorbent foams are conveniently obtainable by foaming an aqueous mixture which, as well as polymerizable and crosslinkable, monoethylenically unsaturated monomers which comprise acid groups and are optionally (partly) neutralized, comprises crosslinkers, the inorganic pulverulent solid and at least one surfactant, and optionally additives or assistants such as solubilizers, thickeners, stabilizers, fillers, fibers and/or cell nucleators, and subsequently polymerizing and/or crosslinking the foamed mixture.

In addition, the superabsorbent foams can be produced in a convenient manner by foaming at least one crosslinkable basic polymer, crosslinkers, the inorganic pulverulent solid and at least one surfactant, and optionally additives or assistants such as solubilizers, thickeners, stabilizers, fillers, fibers and/or cell nucleators as a mixture, and then crosslinking the basic polymers present in the foamed mixture to form a foamable hydrogel.

The foam is optionally subsequently treated with a complexing agent and/or swell retardant. For the sake of simplicity, in the description of measures or properties which are not specific to mixtures which comprise crosslinkable basic polymers or polymerizable and crosslinkable monomers bearing acid groups, even to mixtures which comprise crosslinkable basic polymers but no polymerizable and crosslinkable monomers bearing acid groups, the term “polymerizable aqueous mixture” or even simply “monomer mixture” is also used.

To obtain graphics on the surface of the superabsorbent foam, it is polymerized in a mold which has graphics on at least one internal surface. On this internal surface, these graphics differ from the remaining regions of the internal surface by a different chemical composition or different physical surface properties. The internal surface of the mold is the surface of the mold whose surface is in contact with the monomer mixture or the foam in the course of polymerization of the monomer mixture to give the superabsorbent foam. “Mold” is understood to mean any spatial delimitation of the volume in which the polymerization takes place. This need not necessarily be a closed mold, but rather may also be a temporary carrier material, as is the case, for instance in a belt reactor for polymerization. The “internal surface” of the mold is any surface which is in contact with polymerizing foam and, as a result, imparts a particular geometric shape thereto at the contact surface.

It is suspected that these graphics formed on the internal surface of the mold influence the polymerization of the foam to a locally limited degree such that corresponding graphics form visibly on the foam and are maintained in the course of demolding and through-polymerization of the superabsorbent foam. Graphics applied to the mold by means of ink or paint, however, are also reproduced on the foam, unless they run on to it.

In a simple embodiment of the process according to the invention, the monomer mixture is polymerized in a mold which, on at least one internal surface, has graphics which are formed by sites on the internal surface with different roughness than the remaining internal surface, i.e. have different physical surface properties.

In another embodiment, the monomer mixture is polymerized in a mold, which, on at least one internal surface, has graphics which have been obtained by application of ink or paint, i.e. have a different chemical composition of the surface than the remaining internal surface of the mold. In a further embodiment, the monomer mixture is polymerized in a mold which, on at least one internal surface has graphics which have been obtained by chemical modification of the surface, for instance by etching, but also by physical or physicochemical processes such as ion implantation or deposition of chemical compounds from the gas phase.

In a convenient embodiment of the process according to the invention, the internal side of the mold is printed or painted with ink or paint. The graphics printed on are reproduced correspondingly on the foam. Typically, no color transfer from the mold to the foam takes place. To produce comparatively thin rolled material, the foam can, for example, be polymerized between two films, one or both of which are printed or painted. The polymerization can be effected continuously or batchwise.

In an illustrative embodiment of the invention, an aqueous mixture is foamed which comprises
a) from 10 to 80% by weight of monoethylenically unsaturated monomers, which comprise acid groups and have been neutralized to an extent of at least 50 mol %, or a basic crosslinkable polymer;
b) if monoethylenically unsaturated monomers bearing acid groups are used, optionally additionally up to 50% by weight of other monoethylenically unsaturated monomers,
c) from 0.001 to 10% by weight of crosslinkers,
d) if monoethylenically unsaturated monomers are used, additionally initiators,
e) from 0.1 to 20% by weight of at least one surfactant,
f) optionally a solubilizer,
g) optionally thickeners, foam stabilizers, polymerization regulators, fillers, fibers and/or cell nucleators,
   based in each case on the total amount of the aqueous mixture, and
h) from 1 to 50% by weight of inorganic solid, based on the weight of the finished dry foam,
   where the total amount of the individual components of the mixture including the water adds up to 100% by weight.

The inorganic solid is present in the aqueous mixture typically to an extent of at least 0.5% by weight and at most 30% by weight.

The aqueous mixtures can be foamed, for example, by dispersing fine bubbles of a gas inert towards free radicals in the mixture or by dissolving such a gas in the crosslinkable mixture under a pressure of from 2 to 400 bar and then decompressing it to atmospheric pressure. A free-flowing foam is obtained, which can be filled into molds or hardened on a belt. In the case of use of monomers comprising acid groups, and optionally other monoethylenically unsaturated monomers and crosslinkers, the hardening is effected by polymerization, and, in the case of use of basic polymers, with crosslinking.

Superabsorbent foams based on crosslinked polymers comprising acid groups are known, inter alia, from the prior art documents cited: EP 858 478 B1, page 2, line 55 to page 18, line 22, WO 99/44 648 A1 and WO 00/52 087 A1, page 5, line 23 to page 41, line 18, reference being made here explicitly to all of these. Useful monoethylenically unsaturated monomers which comprise acid groups are the monomers and monomer mixtures which are used to produce granular superabsorbent. The preferred monomer is acrylic acid and salts thereof. Further monoethylenically unsaturated monomers are likewise known from the literature with regard to production of granular superabsorbents.

In one embodiment, foams of water-absorbing acidic polymers are used. The water-absorbing acidic polymers used, which are also referred to hereinafter as acidic superabsorbents, may be all hydrogels which are described, for example, in WO 00/63 295 A1, page 2, line 27 to page 9, line 16. They are essentially lightly crosslinked polymers of acidic monomers which, in at least partly neutralized form, have a high water absorption capacity. Examples of such polymers, in each case lightly crosslinked, are crosslinked polyacrylic acids, crosslinked hydrolyzed graft polymers of acrylonitrile on starch, crosslinked graft polymers of acrylic acid on starch, hydrolyzed crosslinked copolymers of vinyl acetate and acrylic esters, crosslinked polyacrylamides, hydrolyzed crosslinked polyacrylamides, crosslinked copolymers of ethylene and maleic anhydride, crosslinked copolymers of isobutylene and maleic anhydride, crosslinked polyvinylsulfonic acids, crosslinked polyvinylphosphonic acids and crosslinked sulfonated polystyrene. Preference is given to using, as acidic superabsorbents, polymers of (partly) neutralized, lightly crosslinked polyacrylic acid. The (partial) neutralization of the acid groups of the acidic superabsorbents is effected preferably with sodium hydroxide solution, sodium hydrogen carbonate or sodium carbonate. The neutralization can, however, also be undertaken with potassium hydroxide solution, ammonia, amines or alkanolamines such as ethanolamine, diethanolamine or triethanolamine.

Acidic superabsorbents are known from the above references, including from WO 00/63 295 A1, page 2, line 27 to page 9, line 16, to which reference is made explicitly. They may optionally be surface postcrosslinked; to this end, for example, lightly crosslinked polyacrylic acids are reacted with compounds which have at least two groups reactive toward carboxyl groups. These are known crosslinkers. Of particular interest for the application as surface postcrosslinkers are, for example, polyhydric alcohols such as propylene glycol, butanediol-1,4 or hexanediol-1,6 and glycidyl ethers of ethylene glycol and polyethylene glycols with molar masses of from 200 to 1500, preferably from 300 to 400 daltons, and fully acrylated or methacrylated reaction products of trimethylolpropane, of reaction products of trimethylolpropane and ethylene oxide in a molar ratio of 1:1 to 25, preferably 1:3 to 15, and of reaction products of pentaerythritol with ethylene oxide in a molar ratio of 1:30, preferably 1:4 to 20. If it is carried out, the postcrosslinking of the surface of the anionic superabsorbents is carried out, for example, at temperature of up to 220° C., for example, preferably at from 120 to 190° C.

### Crosslinkable Basic Polymers

The production of superabsorbent foams from crosslinkable basic polymers is known, including from WO 03/066 716 A1, to which reference is made here explicitly.

### Further Monomers

A polymerizable aqueous mixture may, as well as monomers bearing acid groups, also comprise further ethylenically unsaturated monomers. Suitable ethylenically unsaturated monomers are, for example acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

### Crosslinkers

Here, in connection with the polymerization of a monomer mixture (or alternatively with the crosslinking of basic polymers), “crosslinker” is understood to mean the crosslinker typically known as “internal crosslinker” in the case of granular superabsorbents, which crosslinks the polymer chains of the superabsorbent to one another, typically achieving—apart from the effect of diffusion of the crosslinker during the crosslinking polymerization or the crosslinking of the polymer chains—an essentially homogeneous degree of crosslinking over the volume of the superabsorbent. Superabsorbents are, however, often also postcrosslinked on the surface with a surface crosslinker, which increases the degree of crosslinking at the surface relative to that in the volume.

Crosslinkers for basic polymers for producing superabsorbent foams, like the foams themselves, are known, inter alia, from WO 03/066 716 A1, to which reference is made here explicitly.

Crosslinkers for polymers based on monomers bearing acid groups are likewise known. The crosslinkers which are also known for the production of granular superabsorbents are used. These are compounds having at least two polymerizable groups which can be polymerized into the polymer network via free-radical mechanism. Suitable crosslinkers are, for example, ethylene glycol dimethacrylate, diethylene glycol diacrylate, allyl methacrylate, trimethylolpropane triacrylate, triallylamine, tetraallyloxyethane, di- and triacrylates, mixed acrylates which, as well as acrylate groups, comprise further ethylenically unsaturated groups, or crosslinker mixtures.

Suitable crosslinkers are especially N,N′-methylenebisacrylamide and N,N′-methylenebismethacrylamide, esters of unsaturated mono- or polycarboxylic acids of polyols, such as diacrylate or triacrylate, for example butanediol diacrylate or dimethacrylate or ethylene glycol diacrylate or dimethacrylate and also trimethylolpropane triacrylate and allyl compounds, such as allyl(meth)acrylate, triallyl cyanurate, diallyl maleate, polyallyl esters, tetraallyloxyethane, triallylamine, tetraallylethylenediamine, allyl esters of phosphoric acid and vinylphosphonic acid derivatives. Further suitable crosslinkers b) are pentaerythritol diallyl ether, pentaerythritol triallyl ether and pentaerythritol tetraallyl ether, polyethylene glycol diallyl ether, ethylene glycol diallyl ether, glyceryl diallyl ether and glyceryl triallyl ether, polyallyl ethers based on sorbitol, and ethoxylated variants thereof. In the process according to the invention, it is possible to use di(meth)acrylates of polyethylene glycols, the polyethylene glycol used having a molecular weight between 300 and 1000.

Particularly advantageous crosslinkers are, however, di- and triacrylates of 3- to 15-tuply ethoxylated glycerol, of 3- to 15-tuply ethoxylated trimethylolpropane, of 3- to 15-tuply ethoxylated trimethylolethane, especially di- and triacrylates of 2- to 6-tuply ethoxylated glycerol or trimethylolpropane, of 3-tuply propoxylated glycerol or trimethylolpropane, and of 3-tuply mixed ethoxylated or propoxylated glycerol or trimethylolpropane, of 15-tuply ethoxylated glycerol or trimethylolpropane, and of 40-tuply ethoxylated glycerol, trimethylolethane or trimethylolpropane.

Very particularly preferred crosslinkers are the polyethoxylated and/or propoxylated glycerols which have been esterified with acrylic acid or methacrylic acid to give di- or triacrylates, as described, for example in WO 03/104 301 A1. Particularly advantageous are di- and/or triacrylates of 3- to 10-tuply ethoxylated glycerol. Very particular preference is given to di- or triacrylates of 1- to 5-tuply ethoxylated and/or propoxylated glycerol. Most preferred are the triacrylates of 3- to 5-tuply ethoxylated and/or propoxylated glycerol.

### Initiators

The initiators used for the polymerization reaction may be all compounds which decompose to free radicals under the polymerization conditions, for example, peroxides, hydroperoxides, hydrogen peroxide, persulfates, azo compounds and the so-called redox initiators, and also any other known method of generating free radicals, for example high-energy radiation, for instance UV light. Preference is given to the use of water-soluble initiators or UV light. In some cases, it is advantageous to use mixtures of different polymerization initiators, for example mixtures of hydrogen peroxide and sodium peroxodisulfate or potassium peroxodisulfate. Mixtures of hydrogen peroxide and sodium peroxodisulfate can be used in any ratio. Suitable organic peroxides are, for example, acetyl acetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl)peroxydicarbonate, dicyclohexyl peroxydicarbonate, di(4-tert-butylcyclohexyl)peroxydicarbonate, dimyristyl peroxydicarbonate, diacetyl peroxydicarbonate, allyl perester, cumyl peroxyneodecanoate, tert-butyl per-3,5,5-trimethylhexanoate, acetylcyclohexylsulfonyl peroxide, dilauryl peroxide, dibenzoyl peroxide and tert-amylperneodecanoate. Further suitable polymerization initiators are azo initiators, e.g. 2,2′-azobis(2-amidinopropane) dihydrochloride, 2,2′-azobis(N,N-dimethylene)isobutyramidine dihydrochloride, 2-(carbamoylazo)isobutyronitrile, 2,2′-azobis[2-(2′-imidazoline-2-yl)propane]dihydrochloride and 4,4′-azobis(4-cyanovaleric acid). The polymerization initiators mentioned are used in customary amounts, for example in amounts of generally at least 0.01 mol %, preferably at least 0.05 mol % and more preferably at least 1 mol %, and generally at most 5 mol %, preferably at most 2 mol %, based on the monomers to be polymerized.

The redox initiators comprise, as the oxidizing component, at least one of the above-specified per compounds and a reducing component, for example, ascorbic acid, glucose, sorbose, ammonium hydrogensulfite, sulfite, thiosulfate, hyposulfite, pyrosulfite or sulfide, or alkali metal hydrogensulfite, sulfite, thiosulfate, hyposulfite, pyrosulfite or sulfide, metal salts such as iron(II) ions or silver ions, or sodium hydroxymethylsulfoxylates. Preference is given to using, as the reducing component of the redox initiator, ascorbic acid, sodium sulfite or sodium pyrosulfite. Based on the amount of monomers used in the polymerization, generally at least 3·10⁻⁶ mol %, preferably from at least 1·10⁻⁵ to 1 mol %, of the reducing component of the redox initiator, and generally at least 1·10⁻⁵, preferably from at least 1·10⁻³ to 5 mol %, of the oxidizing component are used. Instead of the oxidizing component or in addition, it is also possible to use one or more water-soluble azo initiators.

In one embodiment of the invention, a redox initiator consisting of hydrogen peroxide, sodium peroxodisulfate and ascorbic acid is used. For example, these components are used in the concentrations of 1·10⁻² mol % of hydrogen peroxide, 0.084 mol % of sodium peroxodisulfate and 2.5·10⁻³ mol % of ascorbic acid, based on the monomers.

The polymerization can, however, even in the absence of initiators of the type specified above, be triggered by the action of high-energy radiation in the presence of photoinitiators. These may, for example, be so-called alpha-splitters, H-abstracting systems or else azides. Examples of such initiators are benzophenone derivatives such as Michler's ketone, phenanthrene derivatives, fluorene derivatives, anthraquinone derivatives, thioxanthone derivatives, coumarin derivatives, benzoin ethers and derivatives thereof, azo compounds such as the abovementioned free radical formers, substituted hexaarylbisimidazoles or acylphosphine oxides. Examples of azides are: 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulfone, N-(4-sulfonylazidophenyl)maleiimide. N-acetyl-4-sulfonylazidoaniline, 4-sulfonylazidoaniline, 4-azidoaniline, 4-azidophenacyl bromide, p-azidobenzoic acid, 2,6-bis(p-azidobenzylidene)cyclohexanone and 2,6-bis(p-azidobenzylidene)-4-methylcyclohexanone. The photoinitiators are, if they are used, employed typically in amounts of from 0.01 to 5% by weight based on the monomers to be polymerized.

The aqueous monomer solution may comprise the initiator in dissolved or dispersed form. However, the initiators can also be fed to the polymerization reactor separately from the monomer solution.

### Surfactants

The polymerizable or crosslinkable aqueous mixtures comprise, as a further component, from 0.1 to 20% by weight of at least one surfactant. The surfactants are of crucial significance for the production and stabilization of the foam. It is possible to use anionic, cationic or nonionic surfactants or surfactant mixtures which are compatible with one another. It is possible to use low molecular weight or else polymeric surfactants, and combinations of different types or else of the same type of surfactants have been found to be advantageous. Usable nonionic surfactants are, for example, addition products of alkylene oxides, especially ethylene oxide, propylene oxide and/or butylene oxide, onto alcohols, amines, phenols, naphthols or carboxylic acids. Advantageously, the surfactants used are addition products of ethylene oxide and/or propylene oxide on alcohols comprising at least 10 carbon atoms, the addition products comprising from 3 to 200 mol of ethylene oxide and/or propylene oxide added on per mole of alcohol. The addition products comprise the alkylene oxide units in the form of blocks or in random distribution. Examples of usable nonionic surfactants are the addition products of 7 mol of ethylene oxide onto 1 mol of tallow fat alcohol, reaction products of 9 mol ethylene oxide with 1 mol of tallow fat alcohol and addition products of 80 mol of ethylene oxide onto 1 mol of tallow fat alcohol. Further usable commercial nonionic surfactants consist of reaction products of oxo alcohols or Ziegler alcohols with from 5 to 12 mol of ethylene oxide per mole of alcohol, especially with 7 mol of ethylene oxide. Further usable commercial nonionic surfactants are obtained by ethoxylating castor oil. For example, from 12 to 80 mol of ethylene oxide are added on per mole of castor oil. Further usable commercial products are, for example, the reaction products of 18 mol of ethylene oxide with 1 mol of tallow fat alcohol, the addition products of 10 mol of ethylene oxide onto 1 mol of a C₁₃/C₁₅ oxo alcohol, or the reaction products of from 7 to 8 mol of ethylene oxide onto 1 mol of a C₁₃/C₁₅ oxo alcohol. Further suitable nonionic surfactants are phenol alkoxylates, for example, p-tert-butylphenol, which has been reacted with 9 mol of ethylene oxide, or methyl ethers of reaction products formed from 1 mol of a C₁₂ to C₁₈ alcohol and 7.5 mol of ethylene oxide.

The above-described nonionic surfactants can, for example, be converted to the corresponding sulfuric monoesters by esterification with sulfuric acid. The sulfuric monoesters are used as anionic surfactants in the form of the alkali metal or ammonium salts. Suitable anionic surfactants are, for example, alkali metal or ammonium salts of sulfuric monoesters of addition products of ethylene oxide and/or propylene oxide onto fatty alcohols, alkali metal or ammonium salts of alkylbenzenesulfonic acid or of alkylphenol ether sulfates. Products of the type mentioned are commercially available. For example, the sodium salt of a sulfuric monoester of a C₁₃/C₁₅ oxo alcohol, which has been reacted with 106 mol of ethylene oxide, the triethanolamine salt of dodecylbenzenesulfonic acid, the sodium salt of alkylphenol ether sulfates and the sodium salt of sulfuric monoester of a reaction product of 106 mol of ethylene oxide with 1 mol of tallow fat alcohol are commercially usable anionic surfactants. Further suitable anionic surfactants are sulfuric monoesters of C₁₃/C₁₅ oxo alcohols, paraffinsulfonic acids such as C₁₅-alkylsulfonate, alkyl-substituted benzenesulfonic acids and alkyl-substituted naphthalenesulfonic acids such as dodecylbenzenesulfonic acid and di-n-butylnaphthalenesulfonic acid, and also fatty alcohol phosphates such as C₁₅/C₁₈ fatty alcohol phosphate. The polymerizable aqueous mixture may comprise combinations of an anionic surfactant and an anionic surfactant, or combinations of anionic surfactants or combinations of anionic surfactants. Cationic surfactants are also suitable. Examples thereof are the reaction products, quaternized with dimethyl sulfate, of 6.5 mol of ethylene oxide with 1 mol of oleylamine, distearyldimethylammonium chloride, lauryltrimethylammonium chloride, cetylpyridinium bromide and stearic acid triethanolamine ester quaternized with dimethyl sulfate, which is used with preference as the cationic surfactant.

The surfactant content of the aqueous mixture is preferably from 0.5 to 10% by weight. In most cases, the aqueous mixtures have a surfactant content of from 1.5 to 8% by weight.

### Solubilizers

The crosslinkable aqueous mixtures may optionally comprise at least one solubilizer as a further component. This shall be understood to mean water-miscible organic solvents, for example, dimethyl sulfoxide, dimethyl formamide. N-methylpyrrolidone, monohydric alcohols, glycols, polyethylene glycols or monoethers derived therefrom where the monoethers do not comprise any double bonds in the molecule. Suitable ethers are methylglycol, butylglycol, butyldiglycol, methyldiglycol, butyltriglycol, 3-ethoxy-1-propanol and glyceryl monomethyl ether.

The aqueous mixtures comprise from 0 to 50% by weight of a least one solubilizer. If solubilizers are used, their content in the aqueous mixture is preferably from 1 to 25% by weight.

Thickeners, foam stabilizers, fibers, cell nucleators

The crosslinkable aqueous mixture may optionally comprise thickeners, foam stabilizers, fibers and/or cell nucleators. Thickeners are used, for example, to optimize the foam structure and to improve the foam stability. This achieves the effect that the foam shrinks only slightly during the polymerization. Useful thickeners include all natural and synthetic polymers known for this purpose, which greatly increase the viscosity of an aqueous system and do not react with the amino groups of the basic polymers. These may be water-swellable or water-soluble synthetic and natural polymers. A comprehensive overview of thickeners is found, for example, in the publications by R. Y. Lochhead and W. R. Fron, Cosmetics & Toiletries, 108, 95-135 (May 1993) and M. T. Clarke, “Rheological Additives” in D. Laba (ed.) “Rheological Properties of Cosmetics and Toiletries”, Cosmetic Science and Technology Series, Vol. 13, Marcel Dekker Inc., New York 1993.

Water-swellable or water-soluble synthetic polymers useful as thickeners are, for example, high molecular weight polyethylene glycols or copolymers of ethylene glycol and propylene glycol, and also high molecular weight polysaccharides such as starch, guar flour, carob flour or derivatives of natural substances such as carboxymethylcellulose, hydroxyethylcellulose, hydroxymethylcellulose, hydroxypropylcellulose and cellulose mixed ethers. A further group of thickeners is that of water-insoluble products, such as finely divided cellulose powder or other finely divided powders of crosslinked polymers. The aqueous mixtures may comprise the thickeners in amounts of up to 30% by weight. If such thickeners are used at all, they are present in the aqueous mixture in amounts of from 0.1, preferably from 0.5 to 20% by weight.

In order to optimize the foam structure, it is optionally possible to add hydrocarbons having at least 5 carbon atoms in the molecule to the aqueous reaction mixture. Suitable hydrocarbons are, for example, pentane, cyclopentane, hexane, cyclohexane, heptane, octane, isooctane, decane and dodecane. The useful aliphatic hydrocarbons may be straight-chain, branched or cyclic and have a boiling point which is above the temperature of the aqueous mixture during foaming. The aliphatic hydrocarbons increase the lifetime of the as yet unpolymerized foamed aqueous reaction mixture. This eases the handling of the as yet unpolymerized foams and increases the process reliability. The hydrocarbons act, for example, as cell nucleators and simultaneously stabilize the foam already formed. In addition, in the course of polymerization of the monomer foam, they can bring about further foaming of the mixture. In that case, they can also have the function of a blowing agent. Instead of hydrocarbons or in a mixture with them, it is optionally also possible to use chlorinated or fluorinated hydrocarbons as cell nucleators and/or foam stabilizers, for example dichloromethane, trichloromethane, 1,2-dichloroethane, trichlorofluoromethane or 1,1,2-trichlorotrifluoroethane. If hydrocarbons are used, they are used, for example, in amounts of from 0.1 to 20% by weight, preferably from 0.1 to 10% by weight, based on the polymerizable aqueous mixture.

The properties of the foams can optionally also be modified with the aid of further fibers. These may be natural or synthetic fibers or fiber mixtures, for example fibers of cellulose, wool, polyethylene, polypropylene, polyesters or polyamides. If fibers are used, they may be present in the aqueous mixture, for example in an amount up to 200% by weight, preferably up to 25% by weight. Fillers and fibers may optionally also be added to the already foamed mixture. The additional use of fibers leads to an increase in the strength properties, such as wet strength, of the finished foam. The use of fibers in superabsorbent foams is known.

Foams specifically for absorption of salt-containing aqueous solutions

In order to produce foams which have a high absorption capacity also for salt-containing aqueous solutions, the basic and the acidic superabsorbents are used in a mixture, preferably in unneutralized form. The degree of neutralization of the acidic water-absorbing polymers is, for example, from 0 to 100 mol %, preferably from 0 to 75 mol % and usually from 0 to 50 mol %. The water-absorbing basic polymers, in the form of the free bases, have a higher absorption capacity for salt-containing aqueous solutions and especially acidic aqueous solutions than in the form neutralized with acid. When basic polymers are used as the sole water-absorbing polymers, the degree of neutralization is, for example, from 0 to 100 mol %, preferably 0 to 60 mol %.

### Production of the Foams

The above-described crosslinkable aqueous mixtures which contain the monomers or the basic polymer, crosslinkers, inorganic powder and surfactant and optionally further components are first foamed. It is possible, for example, to dissolve an inert gas in the crosslinkable aqueous mixture under a pressure of, for example, from 2 to 400 bar, and then to decompress it to atmospheric pressure. The decompression through a die forms a free-flowing foam. The crosslinkable aqueous mixtures can also be foamed by another method, by dispersing fine bubbles of an inert gas therein. The crosslinkable aqueous mixture can be foamed in the laboratory, for example, by foaming the aqueous mixture in a food processor equipped with a wire whisk. The foam production is preferably carried out in an inert gas atmosphere and with inert gases, for example, by admixing with nitrogen or noble gases under standard pressure or elevated pressure, for example, up to 25 bar and subsequent decompression. The consistency of the foams, the size of the gas bubbles and the distribution of the gas bubbles in the foam can be varied within a wide range, for example, through the selection of the surfactants, solubilizers, foam stabilizers, cell nucleators, thickeners and fillers. This allows the density, the open-cell content of the foam and wall thickness of the foam to be adjusted easily. The aqueous mixture is preferably foamed at temperatures which are below the boiling point of the constituents of the aqueous mixture, for example at from room temperature up to 100° C., preferably at from 20 to 50° C. However, it is also possible to work at temperatures above the boiling point of the component with the lowest boiling point by foaming the mixture in a vessel sealed pressure-tight. Crosslinkable mixtures are obtained in the form of foams which are free-flowing and stable over a prolonged period. The density of the foamed crosslinkable mixture at a temperature of 20° C. is, for example, from 0.01 to 0.9 g/cm³.

Polymerization and/or Crosslinking of the Foamed Mixture

In the second stage of the process, the monomers are polymerized and/or the basic polymer is crosslinked to form a water-absorbing basic polymer. In the polymerization, for example, at least two compounds comprising ethylenically unsaturated double bonds are used as crosslinkers. The polymerization is carried out in the presence of customary free-radical forming initiators. Crosslinked polymers which are superabsorbent are then obtained.

The originally water-soluble basic polymer becomes water-insoluble through the crosslinking. A hydrogel of a basic polymer is obtained. The crosslinkable foam mixtures are transferred, for example, to suitable molds and heated therein, such that the monomers polymerize or the crosslinkers react with the basic polymer. The foamed material can, for example, be applied in the desired thickness to a temporary carrier material, which is advantageously provided with an anti-adhesive coating. For example, the foam can be applied to a substrate with a doctor blade. Another possibility consists in filling the aqueous foam mixture into molds which likewise preferably have an anti-adhesive coating. To produce graphics on the surface of the superabsorbent foam, the mold has graphics on at least one internal surface, which, on this internal surface, differ from the remaining regions of the internal surface by a different chemical composition or different physical surface properties.

Since the foamed aqueous mixture has a long lifetime, this mixture is also suitable for the production of composite materials. It can, for example, be applied to a permanent carrier material, for example, films of polymers (e.g. films of polyethylene, polypropylene or polyamide) or metals such as aluminum. The foamed aqueous mixture can also be applied to nonwoven fabrics, fluff, tissues, woven fabrics, natural or synthetic fibers or to other foams. In the production of composite materials, it may be advantageous under some circumstances to apply the foam to a support material in the form of particular structures or in different layer thickness. However, it is also possible to apply the foam to fluff layers or nonwoven materials and to impregnate these materials such that the fluff, after the crosslinking, is an integral part of the foam. The foamed aqueous mixture obtainable in the first process stage can also be shaped to large blocks and crosslinked. After the crosslinking, the blocks can be cut or sawn to smaller shaped bodies. It is also possible to produce sandwich-type structures by applying a foamed aqueous mixture to a substrate, covering the foam layer with a film or nonwoven fabrics, tissues, woven fabrics, fibers or other foams, and crosslinking the sandwich-type structure by heating. However, it is also possible, before or after the crosslinking, to apply at least one further layer of a foamed, crosslinkable layer, and optionally to cover it with a further film, nonwoven fabrics, tissues, woven fabrics, fibers or other materials. In the second process stage, the composite is then subjected to crosslinking. However, it is also possible to produce sandwich-type structures with further foam layers of the same or different density.

Inventive foam layers with a thickness of up to about 1 millimeter are produced, for example, by heating one side or especially by irradiating one side of the foamed polymerized or crosslinkable aqueous mixture. If thicker layers of a foam are to be produced, for example, foams with thicknesses of several centimeters, the heating of the crosslinkable foamed material with the aid of microwaves is particularly advantageous, because it is possible in this way to achieve relatively homogeneous heating. The crosslinking is effected, for example, at temperatures of from 20 to 180° C., preferably in the range from 40° C. to 160° C., especially at temperatures of from 65 to 140° C. In the case of thicker foam layers which are to be crosslinked, the foamed mixture is heat-treated on both sides, for example, with the aid of contact heating or by means of irradiation. The density of the hydrogel foam corresponds essentially to the density of the crosslinkable aqueous mixture. Foams are thus obtained from water-absorbing polymers with a density of, for example, from 0.01 to 0.9 g/cm³, preferably from 0.1 to 0.7 g/cm³. The polymer foams are open-cell. The open cell content is, for example, at least 80%, and is preferably above 90%. Particular preference is given to foams with an open cell content of 100%. The open cell content in the foam is determined, for example, with the aid of scanning electron microscopy.

Preference is given to foams which are obtainable by proceeding from a polymerizable aqueous mixture which comprises acrylic acid neutralized to an extent of at least 50% with sodium hydroxide solution or potassium hydroxide solution, a crosslinker comprising at least two ethylenically unsaturated double bonds, an initiator, superabsorbent fibers composed of a hydrolyzed and subsequently crosslinked copolymer of isobutene and maleic anhydride and at least one surfactant. Further examples of superabsorbent foams are obtainable by foaming a polymerizable aqueous mixture which comprises at least one basic polymer from the group of polymers comprising vinylamine units, polymers comprising vinylguanidine units, polymers comprising dialkylaminoalkyl(meth)acrylamide units, polyethylenimines, polyamidoamines grafted with ethylenimine, and polydiallyldimethylammonium chlorides.

Foams with a particularly high water absorption capacity and an improved absorption capacity for electrolyte-containing aqueous solutions are obtainable by crosslinking foamed aqueous mixtures of basic polymers which, based on the polymer mixture, comprise from 10 to 90% by weight of a finely divided, water-absorbing, acidic polymer. The acidic hydrogel may be present in the inventive foams as a solid particulate polymer or as a foamed particulate polymer with particle sizes of, for example, from 10 to 2000 μm.

After the crosslinking of the foamed mixture or during the crosslinking, the hydrogel foam is dried. This removes water and other volatile constituents from the crosslinked hydrogel foam. The drying is effected preferably after the crosslinking of the hydrogel foam. Examples of suitable drying processes are thermal convection drying, for example, tray drying, chamber drying, duct drying, flat sheet drying, pan drying, rotary drum drying, freefall tower drying, foraminous belt drying, flow drying, moving bed drying, paddle drying and ball bed drying, thermal contact drying such as hot plate drying, drum drying, belt drying, foraminous drum drying, screw drying, tumble drying and contact disk drying, radiative drying, for example, infrared drying, dielectric drying, for example microwave drying and freeze drying. In order to prevent undesired decomposition and crosslinking reactions, it may be advantageous to carry out the drying at reduced pressure, under a protective gas atmosphere and/or under gentle thermal conditions under which the product temperature does not exceed 120° C., preferably 100° C. Particularly suitable drying processes are (vacuum) belt drying and (if a crushed foam is desired or tolerable) paddle drying.

After the drying step, the hydrogel foam usually no longer comprises any water. The water content of the foamed material can, however, be adjusted as desired by moistening the foam with water or water vapor. Usually, the water content of the gel foam is from 1 to 60% by weight, preferably from 2 to 10% by weight. With the aid of the water content, the flexibility of the hydrogel foam can be adjusted. Fully dried hydrogel foams are hard and brittle, while foamed materials with a water content of, for example, from 5 to 20% by weight are flexible. The foamed hydrogels can be used either directly in the form of films or granules, or individual slabs or films can be cut out of thicker foam blocks.

The above-described hydrogel foams can, however, also be modified to the effect that the surface of the foamed materials is postcrosslinked. This allows the gel stability of the shaped bodies of the foamed hydrogels to be improved. In order to carry out surface postcrosslinking, the surface of the shaped bodies of the foamed hydrogels is treated with at least one crosslinking agent, usually in the form of a solution (the “surface crosslinker”, “postcrosslinker” or “surface postcrosslinker”) and the shaped bodies thus treated are heated to a temperature at which these crosslinkers react with the as yet unpostcrosslinked superabsorbent foam.

Suitable postcrosslinking agents are, for example:
di- or polyepoxides, for instance di- or polyglycidyl compounds such as phosphonic acid diglycidyl ether, ethylene glycol diglycidyl ether or bischlorohydrin ethers of polyalkylene glycols,
alkoxysilyl compounds,
polyaziridines, compounds which comprise aziridine units and are based on polyethers or substituted hydrocarbons, for example, bis-N-aziridinomethane,
polyamines or polyamidoamines and their reaction products with epichlorohydrin,
polyols such as ethylene glycol, 1,2-propanediol, 1,4-butanediol, glycerol, methyltriglycol, polyethylene glycols with a mean molecular weight Mw of 200-10 000, di- and polyglycerol, pentaerythritol, sorbitol, the oxethylates of these polyols and the esters thereof with carboxylic acids or of carbonic acid, such as ethylene carbonate or propylene carbonate,
carbonic acid derivatives such as urea, thiourea, guanidine, dicyandiamide, 2-oxazolidinone and derivatives thereof, bisoxazoline, polyoxazolines, di- and polyisocyanates,
di- and poly-N-methylol compounds such as for example methylenebis(N-methylolmethacrylamide) or melamine-formaldehyde resins,
compounds with two or more blocked isocyanate groups, for example trimethylhexamethylene diisocyanate capped with 2,2,3,6-tetramethyl-4-piperidinone.

If required, acidic catalysts, for example p-toluenesulfonic acid, phosphoric acid, boric acid or ammonium dihydrogen phosphate, can be added.

Particularly suitable postcrosslinking agents are di- or polyglycidyl compounds such as ethylene glycol diglycidyl ether, the reaction products of polyamidoamines with epichlorohydrin, 2-oxazolidinone and N-hydroxyethyl-2-oxazolidinone.

The surface postcrosslinking (often also just “postcrosslinking”) is typically carried out in such a way that a solution of the surface postcrosslinker (often also just “postcrosslinker”) is sprayed onto the surface of the foam.

Surface postcrosslinkers are preferably applied to the foam surface in the form of an aqueous solution. The aqueous solution may comprise water-miscible organic solvents, for example, alcohols such as methanol, ethanol and/or i-propanol or ketones such as acetone. The amount of crosslinker which is applied to the surface of the hydrogel foams is, for example, from 0.1 to 5% by weight, preferably from 1 to 2% by weight. The surface postcrosslinking of the hydrogel foams is effected by heating the hydrogel foams treated with at least one crosslinker at a temperature of, for example, from 60 to 120° C., preferably at from 70 to 100° C. After the surface crosslinking, the water content of the foamed hydrogels postcrosslinked on the surface can likewise be adjusted to values of from 1 to 60% by weight.

Aftertreatment of the Foams with Complexing Agents and/or Swell Retardants

### Complexing Agents

In one embodiment of the invention, the stability of the foam, preferably of a foam which has been obtained from a polymerizable mixture comprising monomers bearing acid groups, is increased by forming complexes on the foam surface. The complexes on the foam are formed by treatment with at least one complexing agent. A complexing agent is an agent which comprises complexing cations. This is preferably brought about by spraying on solutions of divalent or polyvalent cations, in which case the cations can react with functional groups, for instance the acid groups of the polymeric foam, to form complexes. Examples of divalent or polyvalent cations are polymers formed, in a formal sense, completely or partly from vinylamine monomers, such as partly or fully hydrolyzed polyvinylamide (known as “polyvinylamine”), whose amine groups are always—even at very high pH—present partly protonated to ammonium groups or metal cations such as Mg²⁺, Ca²⁺, Ala³⁺, Sc³⁺, Ti⁴⁺, Mn²⁺, Fe^{2+/3+}, Co²⁺, Ni²⁺, Cu³⁺, Zn²⁺, Y³⁺, Zr⁴⁺, La³⁺, Ce⁴⁺, Hf⁴⁺ and Au³⁺. Preferred metal cations are Mg²⁺, Ca²⁺, Al³⁺, Ti⁴⁺, Zr⁴⁺ and La³⁺, and particularly preferred metal cations are Al³⁺, Ti⁴⁺ and Zr⁴⁺. The metal cations may be used either alone or in a mixture with one another. The anions are not subject to any significant restriction; among the metal cations mentioned suitable metal salts are all of those which have a sufficient solubility in the solvent to be used. Particularly suitable metal salts are those with weakly complexing anions, for example, chloride, nitrate and sulfate, hydrogensulfate, carbonate, hydrogencarbonate, nitrate, phosphate, hydrogenphosphate, dihydrogenphosphate and carboxylate, such as acetate and lactate. Particular preference is given to using aluminum sulfate Al₂(SO₄)₃. The solvents used for the metal salts may be water, alcohols, DMF. DMSO and mixtures of these components. Particular preference is given to water and water/alcohol mixtures, for example water/methanol, water/1,2-propanediol and water/1,3-propanediol. Very particular preference is given to water.

The concentration of the polyvalent metal ion in aqueous solution is generally at least 1% by weight, preferably at least 2% by weight, and generally at most 20% by weight, preferably at most 10% by weight. Generally at least 0.05% by weight, preferably at least 0.1% by weight, more preferably at least 0.2% by weight, for example at least 0.8% by weight, and generally at most 10% by weight, preferably at most 8% by weight and more preferably at most 5% by weight, for example at most 3.2% by weight, of the polyvalent metal ion is used, based on the dry foam before application of the complexing agent or swell retardant. In the context of this invention, a foam which comprises 5% by weight of water is “dry”. In the case of use of aluminum sulfate, a content of 0.8% by weight of the cation corresponds to a content of Al₂(SO₄)₃ of 5% by weight, and a content of 3.2% by weight of the cation to a content of Al₂(SO₄)₃ of 20% by weight.

### Swell Retardants

Optionally, the foam is treated with at least one swell retardant. Swell retardants are understood to mean substances which are applied to the surface of the superabsorbent foam in the moist or dry state (<10% by weight of water in the foam) leading to retardation of liquid absorption, which may be advantageous in some applications. The treatment of superabsorbents with swell retardants is known.

### Use of the Foams

The inventive hydrogel foams which are optionally surface postcrosslinked can be used for all purposes for which for example the hydrogel foams based on polymers comprising acid groups, such as crosslinked polyacrylates, known from EP 858 478 B1 are used. The inventive hydrogel foams are suitable, for example, for use in hygiene articles for absorption of body fluids, in bandage material for covering wounds, as a sealing material, as a packaging material, as a soil improver, as a soil replacement, for dewatering sludges, for absorbing acidic aqueous wastes, for thickening aqueous lacquers in the disposal of residual amounts of lacquers, for dewatering water-containing oils or hydrocarbons, or as a material for filters in ventilation systems.

The invention further provides hygiene articles which comprise the inventive foams, such as diapers, sanitary napkins, incontinence articles and bandage material. In hygiene articles, for example, the inventive foams fulfill several functions, specifically acquisition, distribution and/or storage of body fluids. In particular, they are suitable for storing body fluids as a storage layer in a diaper or a feminine hygiene article. By virtue of the retardation of liquid absorption, the liquid to be absorbed is first distributed homogeneously in the hygiene article. It is thus possible, in the case of a second or multiple contacting with liquid offset in time, for the entire area of the foam still to be available to a better degree.

Constructions of hygiene articles which comprise absorbent foam layers are known, as is their production. The inventive absorbent foams are used in these hygiene articles just like known absorbent foams. Foam layers of the inventive hydrogel foams may, for example, in a thickness of from 1 to 5 mm, be arranged within one of the abovementioned hygiene articles as an absorbent core between an upper liquid-pervious cover sheet and a lower liquid-impervious sheet of a film of, for example, polyethylene or polypropylene. The liquid-pervious layer of the hygiene article is in direct contact with the skin of the user in use. This material typically consists of a nonwoven fabric composed of natural fibers such as cellulose fibers or fluff. Optionally, a tissue layer is also arranged above and/or below the absorbent core. Between the lower layer of the hygiene article and the absorbent core, it is optionally also possible for a storage layer of a conventional particulate anionic superabsorbent to be present. When the foamed basic hydrogels are used as the absorbent core in diapers, owing to the open-cell structure of the foamed basic hydrogels, the body fluid which is normally applied all at once in individual amounts is removed rapidly. This imparts a pleasant feeling of surface dryness of the diaper to the user.

### Determination Methods

### Centrifuge Retention Capacity (“CRC”)

In this method, the retention capacity of the superabsorbent foam is determined in a teabag against gravity in g of liquid per g of superabsorbent foam. To determine the CRC, 0.2000+/−0.0050 g of the superabsorbent foam to be tested is introduced in a teabag 60×85 mm in size, which is subsequently sealed shut. Ideally, the foam is dry, i.e. anhydrous, since residual moisture lowers the water absorption of the foam and hence accordingly the CRC. Since the CRC is, however, typically a value of at least 3 g/g and is often even significantly higher, a certain water content of the foam does not lead to noticeable measurement errors. Water contents up to 5% by weight, based on the weight of the moist foam, are tolerable without any problem, usually even higher water contents. The CRC values measured can, if required, also be corrected arithmetically by the water content of the foam.

The teabag is introduced into an excess of 0.9% by weight of sodium chloride solution (at least 0.83 l of sodium chloride solution/1 g of polymer) for 30 minutes. The teabag is then centrifuged at 250 g for 3 minutes. The amount of liquid absorbed and also retained against gravity is determined by weighing the centrifuged teabag. In the same way, a teabag not filled with superabsorbent foam is used to determine a blank value which is subtracted from the amount of liquid absorbed and retained which has been determined with superabsorbent foam. At the starting weight used here, the CRC is calculated simply by multiplying the value thus corrected by the blank value by 5.

### Water Content

The water content is determined by the EDANA (European Disposables and Nonwovens Association, Avenue Eugéne Plasky 157, 1030 Brussels, Belgium; www.edana.org) recommended method 430.2-02, obtainable therefrom.

### EXAMPLES

### Production of the Foams Used in the Examples

In a beaker, with the aid of a magnetic stirrer, the following components were mixed:
209.13 g of acrylic acid
81.31 g of a 37.3% sodium acrylate solution in water
16.8 g of polyethylene glycol diacrylate-400
25.60 g of a 15% aqueous solution of an addition product of 80 mol of ethylene oxide onto 1 mol of a linear saturated C₁₆-C₁₈ fatty alcohol
26.62 g of water and the amounts, specified in the table below, of the inorganic solid powders specified below.

To this suspension are slowly added, with ice cooling, 240.54 g of triethanolamine and the mixture was then allowed to cool to 15° C. The resulting solution was transferred to a pressure vessel and saturated there with carbon dioxide at a pressure of 12 bar by passing a carbon dioxide stream of 3001/h through for 25 min. Under pressure, 16 g of a 3% by weight aqueous solution of 2,2′-azobis(2-amidinopropane) dihydrochloride was added, and then carbon dioxide was passed through the reaction mixture for a further 5 min. The reaction mixture was then pressed at a pressure of 12 bar through a die with a diameter of 1.0 mm to form a fine-cell, readily free-flowing foam.

The resulting monomer foam was applied to a DIN A3-size glass plate with 3 mm-high edges, which had first been covered with a siliconized polyester film (siliconized polyethylene terephthalate film Hostaphan® RN, thickness 36 μm, obtainable from Mitsubishi Polyester Film GmbH, Wiesbaden, Germany). The foam is covered at the top with a piece of film of the same type, to which graphics (a pattern) had been applied with a commercial pen for writing on plastics (“film pen”). The upper film is covered with a second glass plate. The foam sample was irradiated with UV light synchronously from both sides over 4 minutes, from above with a UVASPOT 1000/T UV/VIS radiator from Dr. Hönle A G, Gräfelfing, Germany, and from below with 2 UVASPOT 400/T UV/VIS radiators from the same manufacturer.

The resulting foam layer was dried completely under a nitrogen stream in a vacuum drying cabinet at 80° C., and then adjusted to a moisture content of 5% by weight by spraying with water.

The results with different fillers and amounts of fillers are compiled in the table which follows. The amount of the filler in the foam was converted to dry foam. The appearance of graphics on the foam surface was assessed visually. The “graphics” column states how well graphics were reproduced:

nd not determined
0 no appearance of graphics
0+ weak appearance of graphics
+ good appearance of graphics
++ very good appearance of graphics

| | Filler | | | |
|---|---|---|---|---|
| | | Amount in | Amount in | |
| | | monomer | the foam | |
| Example # | Type | solution [g] | [% by wt.] | Graphics |
| 1 | Kaolin | 4.8 | 1 | 0+ |
| 2 | ″ | 24 | 4.6 | 0+ |
| 3 | ″ | 48 | 8.7 | + |
| 4 | ″ | 96 | 16.1 | + |
| 5 | ″ | 96 | 16.1 | ++ |
| 6 | Talc | 4.8 | 1 | nd |
| 7 | ″ | 24 | 4.6 | nd |
| 8 | ″ | 48 | 8.7 | 0 |
| 9 | ″ | 96 | 16.1 | 0 |
| 10 | ″ | 192 | 27.7 | 0+ |
| 11 | TiO₂ | 4.8 | 1 | 0+ |
| 12 | ″ | 24 | 4.6 | ++ |
| 13 | ″ | 48 | 8.7 | ++ |

The examples show that, depending on the solids content and the solids type, foams which bear graphics on their surface are obtained.

1. A superabsorbent foam which comprises at least 1% by weight, based on the total weight of the dry foam, of an inorganic pulverulent solid and bears graphics on at least one of its surfaces.

2. The foam according to claim 1, which comprises at least 5% by weight, based on the total weight of the dry foam, of the inorganic solid.

3. The foam according to claim 1, which is a crosslinked polymer based on monomers bearing acid groups.

4. The foam according to claim 3, which is a crosslinked polymer based on a mixture of acrylic acid and sodium acrylate.

5. The foam according to claim 1, whose surface has been postcrosslinked and/or treated with a complexing agent.

6. The foam according to claim 1, wherein the inorganic pulverulent solid is titanium dioxide.

7. A process for producing a superabsorbent foam which comprises at least 1% by weight, based on the total weight of the dry foam, of an inorganic pulverulent solid and bears graphics on at least one of its surfaces, in which a foamed monomer mixture which comprises at least 1% by weight, based on the total weight of the dry foam, of the inorganic pulverulent solid, and polymerizable and crosslinkable, optionally (partly) neutralized, monoethylenically unsaturated monomers comprising acid groups or at least one crosslinkable basic polymer, and also crosslinkers, inorganic pulverulent filler, and at least one surfactant, is polymerized in a mold which bears graphics on at least one internal surface.

8. The process according to claim 7, wherein the polymerization is followed by treatment of the surface of the foam with a surface postcrosslinking agent and/or a complexing agent.

9. (canceled)

10. A hygiene article for absorbing body fluids, which comprises a foam defined in claim 1.

11. A method of absorbing body fluids comprising contacting the body fluid with a superabsorbent foam of claim 1.
